(19) United States
(12) Patent Application Publication
(10) Pub. No.: US 2021/0077406 A1
(43) Pub. Date: Mar. 18, 2021

(54) COMPOSITION AND METHOD FOR FREEZE-DRYING PHARMACEUTICAL COMPOSITION CONTAINING ANIONIC DRUG

(71) Applicant: SAMYANG BIOPHARMACEUTICALS CORPORATION, Seoul (KR)

(72) Inventors: Ji Hye CHOI, Seongnam-si (KR); Ji Yeon SON, Yongin-si (KR); So Jin LEE, Yongin-si (KR); Bo Mi KIM, Seongnam-si (KR); Hye Yeong NAM, Seongnam-si (KR)

(73) Assignee: SAMYANG BIOPHARMACEUTICALS CORPORATION, Seoul (KR)

(21) Appl. No.: 16/764,643

(22) PCT Filed: Nov. 15, 2018
(86) PCT No.: PCT/KR2018/013964

§ 371 Date: May 15, 2020

(30) Foreign Application Priority Data
Nov. 16, 2017 (KR) 10-2017-0153053

(51) Int. Cl. A61K 9/19; A61K 9/16; A61K 47/69; C12N 15/113

(52) U.S. Cl. CPC A61K 9/19; A61K 9/1623; A61K 9/1617; A61K 47/6949; C12N 15/1135; A61K 9/1635; A61K 9/1641; A61K 9/1682; A61K 9/1647

(57) ABSTRACT

Disclosed are a composition and a method for freeze-drying which allow excellent stability, safety, and efficacy to be exhibited at the time of freeze-drying and reconstituting a composition for anionic drug delivery.

### TECHNICAL FIELD

The present invention relates to a freeze-dried composition of a pharmaceutical composition containing anionic drug and a method for preparing the same, and more specifically, a composition and a method for freeze-drying of a composition for delivering anionic drug such as nucleic acid, and a freeze-dried product thereof.

### BACKGROUND ART

Drugs are usually commercialized as final products in dried formulation. For this, conventional drying processes such as reduced pressure-drying, spray-drying, freeze-drying, etc. can be utilized. Among them, freeze-drying process is a method comprising freezing a material and drying the frozen material by sublimation which makes ice directly to vapor by lowering partial water vapor pressure. In such freeze-drying processes, there are many cases of using a cryoprotectant.

According to the Korean Guidelines on Drug Additives (December 2015), additive is generally added to drug formulation in order to improve the quality and economic feasibility while maintaining stability, safety or homogeneity of the formulation. That is, additive refers to a material which is further used in drug formulation in order to increase usefulness such as stability, safety, quality, etc. Such additives are used in drug formulation to control the quality of drugs, and in general, since there are many cases of using additives in large amounts, their safety must be confirmed especially.

The efficacy of anionic drugs, particularly nucleic acid, in gene therapy has been validated. Thus, safe and efficient drug delivery technologies have been studied for a long time, and various delivery systems and delivery technologies have been developed. Recently, non-viral delivery systems are actively developed. As compared with viral delivery systems, although non-viral delivery systems are less efficient, they have advantages of lower side effect in terms of in vivo safety and lower production cost in terms of economic feasibility. In particular, Korean Patent Publication No. 10-2017-0032858 A discloses a composition for delivering an anionic drug, comprising: an anionic drug as an active ingredient; a cationic compound; an amphiphilic block copolymer; and a salt of polylactic acid; wherein the anionic drug forms a complex with the cationic compound by electrostatic interaction, and the complex is entrapped in a nanoparticle structure formed by the amphiphilic block copolymer and the salt of polylactic acid.

As such, many studies have been conducted on anionic drugs in terms of the efficiency of in vivo and ex vivo delivery, the validation of treatment effect, etc. However, cryoprotectants have not yet been studied. Particularly, nucleic acid-type anionic drugs may exhibit structural changes during freezing or freeze-drying, and may cause structural changes in non-viral delivery systems.

### CONTENTS OF THE INVENTION

### Problems to be Solved

Under the technical background as described above, one object of the present invention is to provide a composition for freeze-drying of a composition for delivering anionic drug, which can allow the composition for delivering anionic drug to show good stability and safety during freeze-drying and reconstitution, and good efficacy.

Another object of the present invention is to provide a method for freeze-drying of a composition for delivering anionic drug, which can allow the composition for delivering anionic drug to show good stability and safety during freeze-drying and reconstitution, and good efficacy.

A further object of the present invention is to provide a freeze-dried product of a composition for delivering anionic drug, which can show good stability and safety during freeze-drying and reconstitution, and good efficacy.

### Technical Means to Solve the Problems

One aspect of the present invention relates to a composition for freeze-drying of a composition for delivering anionic drug, which comprises: a composition for delivering anionic drug comprising an anionic drug, a cationic compound, an amphiphilic block copolymer and a salt of polylactic acid, wherein the anionic drug forms a complex with the cationic compound by electrostatic interaction, the complex is entrapped in a nanoparticle structure formed by the amphiphilic block copolymer and the salt of polylactic acid; and sorbitol as a cryoprotectant.

Another aspect of the present invention relates to a method for freeze-drying of a composition for delivering anionic drug, comprising a step of conducting the freeze-drying by using the above-stated composition for freeze-drying.

A further aspect of the present invention relates to a freeze-dried product of a composition for delivering anionic drug freeze-dried by the above-stated method.

The present invention will be explained in detail below.

The present inventors have added various cryoprotectants to the composition for delivering anionic drug according to Korean Patent Publication No. 10-2017-0032858 A which was filed by the present applicant, and conducted freeze-dryings and reconstitutions of the resulting compositions, and finally confirmed that use of sorbitol provided unexpectedly good stability and safety, and good efficacy. That is, the present inventors have found for the first time that, in freeze-drying and reconstitution, addition of sorbitol could show lower content of unentrapped drug (e.g., siRNA), less disintegration of drug (e.g., siRNA), lower toxicity and higher efficacy at the same time, as compared with the cases of adding other cryoprotectants for example, trehalose, mannitol, sucrose or glucose.

Accordingly, the present invention is characterized in using sorbitol as a cryoprotectant in freeze-drying of a composition for delivering anionic drug.

In an embodiment of the present invention, preferably sorbitol is used in an amount of 1 to 5,000 parts by weight, based on 1 part by weight of the anionic drug. It may be used in an amount of, more preferably 1 to 4,000 parts by weight, still more preferably 5 to 3,000 parts by weight, and most preferably 5 to 2,000 parts by weight. Within the above amount ranges, low content of unentrapped drug, less disintegration of drug, low toxicity and high efficacy can be obtained.

Among the components of the composition for delivering anionic drug, the anionic drug and the cationic compound are entrapped in a nanoparticle structure formed by the amphiphilic block copolymer and the salt of polylactic acid. A schematic structure of the polymer nanoparticle delivery system, in which the anionic drug and the cationic compound are entrapped, is shown in FIG. 1. Referring to FIG. 1, the anionic drug and the cationic compound are combined together through electrostatic interaction between them to form a complex of the anionic drug and the cationic compound. The complex of the anionic drug and the cationic compound as formed is entrapped in a nanoparticle structure formed by the amphiphilic block copolymer and the salt of polylactic acid.

As shown in FIG. 1, the nanoparticle structure formed by the amphiphilic block copolymer and the salt of polylactic acid is a structure wherein, under aqueous environment, the hydrophilic part of the amphiphilic block copolymer forms the outer wall of the nanoparticle, the hydrophobic part of the amphiphilic block copolymer and a salt of polylactic acid-which is contained as a separate ingredient from the amphiphilic block copolymer-form the inner wall of the nanoparticle, and the anionic drug and the cationic compound are entrapped in the formed nanoparticle.

Since the complex of the anionic drug and the cationic compound is maintained in the state entrapped in the nanoparticle structure formed by the amphiphilic block copolymer and the salt of polylactic acid, the stability in blood or body fluid is improved. According to an embodiment, the particle size of the nanoparticle may be 10 to 200 nm, and more specifically, it is 10 to 150 nm. In addition, the standard charge of the nanoparticle may be −20 to 20 mV, and more specifically, it is −10 to 10 mV. The above particle size and the standard charge of the nanoparticle are preferred in terms of stability of the nanoparticle structure, contents of the constitutional ingredients, absorption of the anionic drug in a body, and convenience of sterilization as a pharmaceutical composition.

The anionic drug contained as an active ingredient in the composition according to the present invention may include any pharmacologically active material that takes negative charge in the molecule in an aqueous solution. As a concrete embodiment, the anionic property may be provided from one or more functional groups selected from the group consisting of carboxylic group, phosphate group and sulfate group. In addition, as an embodiment of the present invention, the anionic drug may be a multi-anionic drug such as peptide, protein or heparin, or a nucleic acid.

The nucleic acid may be deoxyribonucleic acid, ribonucleic acid, or a nucleic acid drug such as a polynucleotide derivative in which the backbone, sugar or base is chemically modified or the end is modified. More specifically, it may be a nucleic acid selected from the group consisting of RNA, DNA, siRNA (short interfering RNA), aptamer, antisense ODN (oligodeoxynucleotide), antisense RNA, ribozyme and DNAzyme, etc. In addition, the backbone, sugar or base of the nucleic acid may be chemically modified or its end may be modified for the purpose of increasing stability in blood or weakening immune reactions, and the like. Specifically, a part of phosphodiester bond of nucleic acid may be replaced with phosphorothioate or boranophosphate bond, or 2′-OH positions of a part of ribose bases may include one or more modified nucleotides into which various functional groups such as methyl group, methoxyethyl group, fluorine, etc. are introduced.

Furthermore, one or more ends of the nucleic acid may be modified with one or more selected from the group consisting of cholesterol, tocopherol and C₁₀₋₂₄ fatty acid. In case of siRNA, for example, 5′ or 3′ end, or both ends of the sense and/or antisense strand may be modified, and preferably the end of the sense strand may be modified.

The cholesterol, tocopherol and C₁₀₋₂₄ fatty acid also include analogues, derivatives and metabolites of each of the cholesterol, tocopherol and C₁₀₋₂₄ fatty acid.

The siRNA refers to duplex RNA or single-strand RNA wherein a double-stranded from exists inside the single-strand RNA, which may reduce or inhibit expression of a target gene by mediating degradation of mRNA complementary to the sequence of siRNA when the siRNA exists in the same cell as that of the target gene. The double strands are bound to each other by hydrogen bonding between nucleotides. It is not necessary that all nucleotides in the double strands should be complementarily bound with the corresponding nucleotides, and the both strands may be separated or may not be separated. As an embodiment, the length of the siRNA may be about 15 to 60 nucleotides (which means the number of nucleotides of one side of double-stranded RNA, i.e., the number of base pairs; and in case of a single-stranded RNA, the length of double strands existing inside the single stranded RNA), specifically about 15 to 30 nucleotides, and more specifically about 19 to 25 nucleotides.

As an embodiment, the double-stranded siRNA may have an overhang of 1-5 nucleotides at one or both ends of the 3′ or 5′ end. In another embodiment, it may be blunt without any overhang at both ends. Specifically, it may be siRNA disclosed in US Patent Publication No. 2002/0086356 A1 or U.S. Pat. No. 7,056,704 B2 (incorporated herein by references).

In addition, the siRNA may have a symmetrical structure with the same lengths of two strands, or it may have a non-symmetrical structure with one strand shorter than the other. Specifically, it may be a non-symmetrical siRNA molecule of double strands consisting of an antisense of 19 to 21 nucleotides (nt); and a sense of 15 to 19 nt having a sequence complementary to the antisense, wherein the 5′ end of the antisense is the blunt end, and the 3′ end of the antisense has an overhang of 1-5 nucleotides. Specifically, it may be siRNA disclosed in International Publication No. WO 2009/078685 A2.

In the present invention, the anionic drug is preferably contained in an amount of 0.001 to 10% by weight, more specifically 0.01 to 8% by weight, based on the total weight of the composition. If the amount of the anionic drug is less than 0.001% by weight, the amount of the delivery system used becomes too large as compared with the drug, and thus side effects may be caused by the delivery system. If the amount of the anionic drug is greater than 10% by weight, the size of the nanoparticle becomes too large, and thus the stability of the nanoparticle may be lowered and the rate of loss during filter sterilization may increase.

In a concrete embodiment, the cationic compound is combined with the anionic drug by electrostatic interaction to form a complex, and the complex is entrapped in the nanoparticle structure of the amphiphilic block copolymer. Therefore, the cationic compound may include any type of compound capable of forming a complex with the anionic drug by electrostatic interaction, and for example, it may include lipids and polymers. The cationic lipid may may be one or a combination of two or more selected from the group consisting of N,N-dioleyl-N,N-dimethylammoniumchloride (DODAC), N,N-distearyl-N,N-dimethylammoniumbromide (DDAB), N-(1-(2,3-dioleoyloxy)propyl-N,N,N-trimethylammoniumchloride (DOTAP), N,N-dimethyl-(2,3-dioleoyloxy)propylamine (DODMA), N,N,N-trimethyl-(2,3-dioleoyloxy)propylamine (DOTMA), 1,2-diacyl-3-trimethylammonium-propane (TAP), 1,2-diacyl-3-dimethylammonium-propane (DAP), 3β-[N—(N′,N′,N′-trimethylaminoethane)carbamoyl]cholesterol (TC-cholesterol), 3β-[N—(N′,N′-dimethylaminoethane)carbamoyl]cholesterol (DC-cholesterol), 3β-[N—(N′-monomethylaminoethane)carbamoyl]cholesterol (MC-cholesterol), 3β-[N-(aminoethane)carbamoyl]cholesterol (AC-cholesterol), cholesteryloxypropane-1-amine (COPA), N—(N′-aminoethane)carbamoylpropanoic tocopherol (AC-tocopherol) and N—(N′-methylaminoethane)carbamoylpropanoic tocopherol (MC-tocopherol). If such a cationic lipid is used, it is preferable to use polycationic lipid having high cation density as less as possible in order to decrease toxicity induced by the cationic lipid, and more specifically, the number of the functional group in a molecule which is capable of exhibiting positive charge in an aqueous solution may be one. Accordingly, in a more preferable embodiment, the cationic lipid may be one or more selected from the group consisting of 3β-[N—(N′,N′,N′-trimethylaminoethane)carbamoyl]cholesterol (TC-cholesterol), 3β-[N—(N′,N′-dimethylaminoethane)carbamoyl]cholesterol (DC-cholesterol), 3β-[N—(N′-monomethylaminoethane)carbamoyl]cholesterol (MC-cholesterol), 3β-[N-(aminoethane)carbamoyl]cholesterol (AC-cholesterol), N-(1-(2,3-dioleoyloxy)propyl-N,N,N-trimethylammoniumchloride (DOTAP), N,N-dimethyl-(2,3-dioleoyloxy)propylamine (DODMA) and N,N,N-trimethyl-(2,3-dioleoyloxy)propylamine (DOTMA). On the other hand, the cationic polymer may be selected from the group consisting of chitosan, glycol chitosan, protamine, polylysine, polyarginine, polyamidoamine (PAMAM), polyethylenimine, dextran, hyaluronic acid, albumin, polyethylenimine (PEI), polyamine and polyvinylamine (PVAm), and preferably it may be one or more selected from polyethylenimine (PEI), polyamine and polyvinylamine (PVA).

In a concrete embodiment, the cationic lipid may be a cationic lipid represented by the following Formula 7:

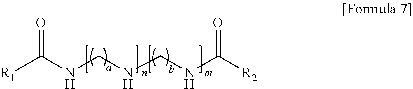

In the above formula,

each of n and m is 0 to 12 with the proviso that 2≤n+m≤12, each of a and b is 1 to 6, and each of R₁ and R₂ is independently selected from the group consisting of saturated and unsaturated C₁₁₋₂₅ hydrocarbons.

Preferably, n and m may be independently 1 to 9, and 2 K n+m K 10.

Preferably, a and b may be 2 to 4.

Preferably, each of R₁ and R₂ may be independently selected from the group consisting of lauryl, myristyl, palmityl, stearyl, arachidyl, behenyl, lignoceryl, cerotyl, myristoleyl, palmitoleyl, sapienyl, oleyl, linoleyl, arachidonyl, eicosapentaenyl, erucyl, docosahexaenyl and cerotyl.

Concrete example of the cationic lipid may be one or more selected from the group consisting of 1,6-dioleoyl triethylene tetramide, 1,8-dilinoleoyl tetraethylene pentamide, 1,4-dimyristoleoyl diethylene triamide, 1,10-distearoyl pentaethylene hexamide and 1,10-dioleoyl pentaethylene hexamide.

The cationic compound used in the present invention may be contained in an amount of 0.01 to 50% by weight, more specifically 0.1 to 10% by weight, based on the total weight of the composition. If the amount of the cationic compound is less than 0.01% by weight, it may not be sufficient to form a complex with the anionic drug. If the amount of the cationic compound is greater than 50% by weight, the size of the nanoparticle becomes too large, and thus the stability of the nanoparticle may be lowered and the rate of loss during filter sterilization may increase.

The cationic compound and the anionic drug are combined together through electrostatic interaction to form a complex. As a concrete embodiment, the ratio of quantities of electric charge of the cationic compound (N) and the anionic drug (P) (N/P: the ratio of the positive electric charge of the cationic compound to the negative electric charge of the anionic drug) is 0.1 to 128, more specifically 0.5 to 64, still more specifically 1 to 32, still more specifically 1 to 24, and most specifically 6 to 24. If the ratio (N/P) is less than 0.1, it may be difficult to form a complex comprising a sufficient amount of anionic drug. Thus, it is advantageous that the ratio (N/P) is 0.1 or greater so that a complex comprising a sufficient amount of anionic drug may be formed. On the other hand, if the ratio is greater than 128, toxicity may be induced. Thus, it is advantageous that the ratio is 128 or less.

In a concrete embodiment, the amphiphilic block copolymer may be an A-B type block copolymer comprising a hydrophilic A block and a hydrophobic B block. The A-B type block copolymer forms a core-shell type polymeric nanoparticle in an aqueous solution, wherein the hydrophobic B block forms the core (inner wall) and the hydrophilic A block forms the shell (outer wall).

In this regard, the hydrophilic A block may be one or more selected from the group consisting of polyalkyleneglycol, polyvinyl alcohol, polyvinyl pyrrolidone, polyacrylamide, and derivatives thereof. More specifically, the hydrophilic A block may be one or more selected from the group consisting of monomethoxy polyethylene glycol, monoacetoxy polyethylene glycol, polyethylene glycol, a copolymer of polyethylene and propylene glycol, and polyvinyl pyrrolidone. The hydrophilic A block may have a number average molecular weight of 200 to 50,000 Daltons, more specifically 1,000 to 20,000 Daltons, and still more specifically 1,000 to 5,000 Daltons.

If necessary, a functional group or a ligand that may bind to a specific tissue or cell, or a functional group capable of promoting intracellular delivery may be chemically conjugated to the end of the hydrophilic A block so as to control the in vivo distribution of the polymeric nanoparticle delivery system formed by the amphiphilic block copolymer and the salt of polylactic acid, or to increase the efficiency of delivery of the nanoparticle delivery system into cells. The functional group or ligand may be one or more selected from the group consisting of monosaccharide, polysaccharide, vitamins, peptides, proteins, and antibody to cell surface receptor. More specifically, the functional group or ligand may be one or more selected from the group consisting of anisamide, vitamin B9 (folic acid), vitamin B12, vitamin A, galactose, lactose, mannose, hyaluronic acid, RGD peptide, NGR peptide, transferrin, antibody to transferrin receptor, etc.

The hydrophobic B block is a biocompatible and biodegradable polymer, and it may be one or more selected from the group consisting of polyester, polyanhydride, polyamino acid, polyorthoester and polyphosphazine. More specifically, the hydrophobic B block may be one or more selected from the group consisting of polylactide, polyglycolide, polycaprolactone, polydioxane-2-one, a copolymer of polylactide and glycolide, a copolymer of polylactide and polydioxane-2-one, a copolymer of polylactide and polycaprolactone, and a copolymer of polyglycolide and polycaprolactone. In another embodiment, the hydrophobic B block may have a number average molecular weight of 50 to 50,000 Daltons, more specifically 200 to 20,000 Daltons, and still more specifically 500 to 5,000 Daltons. In addition, in order to improve the stability of the nanoparticle by increasing hydrophobicity of the hydrophobic block, tocopherol, cholesterol or C₁₀₋₂₄ fatty acid may be chemically conjugated to a hydroxyl group at the end of the hydrophobic block.

The amount of the amphiphilic block copolymer comprising the hydrophilic block (A) and the hydrophobic block (B) is 40 to 99.98% by weight, and preferably, it may be specifically 50 to 99.8% by weight, and more specifically 60 to 90% by weight, based on the total dry weight of the composition. If the amount of the amphiphilic block copolymer is less than 40% by weight, the size of the nanoparticle becomes too large, and thus the stability of the nanoparticle may be lowered and the rate of loss during filter sterilization may increase. If the amount of the amphiphilic block copolymer is greater than 99.98% by weight, the amount of anionic drug that can be incorporated may become too small.

In another embodiment, the amphiphilic block copolymer may comprise 40 to 70% by weight, more specifically 50 to 60% by weight, of the hydrophilic block (A), based on the weight of the copolymer. If the amount of the hydrophilic block (A) is less than 40% by weight, solubility of the polymer in water is low, and thus it may be difficult to form a nanoparticle. Thus, it is advantageous that the amount of the hydrophilic block (A) is 40% by weight or greater so that the copolymer can have a solubility in water sufficient to form a nanoparticle. If the amount of the hydrophilic block (A) is greater than 70% by weight, hydrophilicity becomes too high and thus the stability of the polymeric nanoparticle may be lower and it may be difficult to use as a composition for solubilizing the anionic drug/cationic lipid complex. Thus, considering stability of the nanoparticle, it is advantageous that the amount of the hydrophilic block (A) is 70% by weight or less.

In a concrete embodiment, the amphiphilic block copolymer allows enclosure of the complex of the anionic drug and the cationic lipid in the nanoparticle structure in an aqueous solution, wherein the ratio of the weight of the complex of the anionic drug and the cationic lipid (a) to the weight of the amphiphilic block copolymer (b) [a/b×100; (the weight of the anionic drug+the weight of the cationic lipid)/the weight of the amphiphilic block copolymer×100] may be 0.001 to 100% by weight, specifically 0.01 to 50% by weight, and more specifically 0.1 to 20% by weight. If the weight ratio is less than 0.001% by weight, the amount of the complex of the anionic drug and the cationic lipid become too small, and thus it may be difficult to satisfy the effective amount of the anionic drug for action. If the weight ratio is greater than 100% by weight, a nanoparticle structure of appropriate size may not be formed, considering the molecular weight of the amphiphilic block copolymer and the amount of the complex of the anionic drug and the lipid.

The nanoparticle structure in the composition according to the present invention is characterized in comprising a salt of polylactic acid (e.g. PLANa). The salt of polylactic acid is distributed in the core (inner wall) of the nanoparticle and acts to enhance hydrophobicity of the core and stabilize the nanoparticle, and at the same time, to effectively avoid reticuloendothelial system (RES) in the body. That is, the carboxylic anion in the salt of polylactic acid binds to the cationic complex more efficiently than a polylactic acid, and decreases the surface charge of the polymeric nanoparticle. Thereby, positive charge of the surface potential of the polymeric nanoparticle becomes less than that of a polymeric nanoparticle which does not contain a salt of polylactic acid, and thus it may be less captured by reticuloendothelial system and efficiently delivered to target sites (e.g., cancer cells, inflammatory cells, etc.).

The salt of polylactic acid—which is contained as a separate ingredient from the amphiphilic block copolymer—is a component of the inner wall of the nanoparticle, and may have a number average molecular weight of 500 to 50,000 Daltons, and more specifically 1,000 to 10,000 Daltons. If the number average molecular weight of the salt of polylactic acid is less than 500 Daltons, the hydrophobicity becomes too low and thus the salt of polylactic acid may not easily exist at the core (inner wall) of the nanoparticle. If the number average molecular weight of the salt of polylactic acid is greater than 50,000 Daltons, the size of the polymeric nanoparticle may become too large.

The salt of polylactic acid may be used in an amount of 1 to 200 parts by weight, more specifically 1 to 100 parts by weight, and still more specifically 10 to 60 parts by weight, based on 100 parts by weight of the amphiphilic block copolymer. If the amount of the salt of polylactic acid is greater than 200 parts by weight based on 100 parts by weight of the amphiphilic block copolymer, the size of the nanoparticle increases and thus the sterilized membrane filtration may become difficult. If the amount of the salt of polylactic acid is less than 1 part by weight based on 100 parts by weight of the amphiphilic block copolymer, it is hard to obtain the desired effect.

In an embodiment, the composition of the present invention may comprise 10 to 1,000 parts by weight of the amphiphilic block copolymer and 5 to 500 parts by weight of the salt of polylactic acid, based on 1 part by weight of the anionic drug. Preferably, the amphiphilic block copolymer may be contained in an amount of 50 to 800 parts by weight, and more preferably 100 to 500 parts by weight. Preferably, the salt of polylactic acid may be contained in an amount of 5 to 300 parts by weight, and more preferably 10 to 100 parts by weight.

In an embodiment, the end of the salt of polylactic acid opposite to the end where the salt is formed (e.g., sodium carboxylate) may be substituted with one selected from the group consisting of hydroxyl, acetoxy, benzoyloxy, decanoyloxy, palmitoyloxy, and C₁₋₂ alkoxy.

As a preferred embodiment, the salt of polylactic acid in the present invention may be one or more selected from the group consisting of the compounds of the following Formulas 1 to 6:

RO—CHZ-[A]ₙ-[B]ₘ—COOM [Formula 1]

In Formula 1 above, A is —COO—CHZ—; B is —COO—CHY—, —COO—CH₂CH₂CH₂CH₂CH₂— or —COO—CH₂CH₂OCH₂; R is a hydrogen atom, or acetyl, benzoyl, decanoyl, palmitoyl, methyl or ethyl; each of Z and Y is a hydrogen atom, or methyl or phenyl; M is Na, K or Li; n is an integer of from 1 to 30; and m is an integer of from 0 to 20.

RO—CHZ—[COO—CHX]ₚ—[COO—CHY′]_{g}—COO—CHZ—COOM [Formula 2]

In Formula 2 above, X is methyl; Y′ is a hydrogen atom or phenyl; p is an integer of from 0 to 25, q is an integer of from 0 to 25, with the proviso that p+q is an integer of from 5 to 25; R is a hydrogen atom, or acetyl, benzoyl, decanoyl, palmitoyl, methyl or ethyl; M is Na, K or Li; and Z is a hydrogen atom, methyl or phenyl.

RO-PAD-COO—W-M′ [Formula 3]

In Formula 3 above, W-M′ is

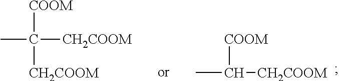

PAD is selected from the group consisting of D,L-polylactic acid, D-polylactic acid, polymandelic acid, copolymer of D,L-lactic acid and glycolic acid, copolymer of D,L-lactic acid and mandelic acid, copolymer of D,L-lactic acid and caprolactone, and copolymer of D,L-lactic acid and 1,4-dioxane-2-one; R is a hydrogen atom, or acetyl, benzoyl, decanoyl, palmitoyl, methyl or ethyl; and M is independently Na, K or Li.

S—O-PAD-COO-Q [Formula 4]

In Formula 4 above, S is

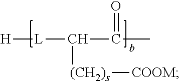

L is —NR₁— or —O—, wherein R₁ is a hydrogen atom or C₁₋₁₀ alkyl; Q is CH₃, CH₂CH₃, CH₂CH₂CH₃, CH₂CH₂CH₂CH₃, or CH₂C₆H₅; a is an integer of from 0 to 4; b is an integer of from 1 to 10; M is Na, K or Li; and PAD is one or more selected from the group consisting of D,L-polylactic acid, D-polylactic acid, polymandelic acid, copolymer of D,L-lactic acid and glycolic acid, copolymer of D,L-lactic acid and mandelic acid, copolymer of D,L-lactic acid and caprolactone, and copolymer of D,L-lactic acid and 1,4-dioxane-2-one.

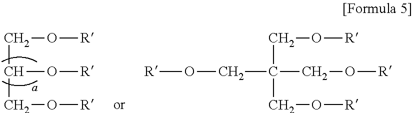

In Formula 5 above, R′ is -PAD-O—C(O)—CH₂CH₂—C(O)—OM, wherein PAD is selected from the group consisting of D,L-polylactic acid, D-polylactic acid, polymandelic acid, copolymer of D,L-lactic acid and glycolic acid, copolymer of D,L-lactic acid and mandelic acid, copolymer of D,L-lactic acid and caprolactone, and copolymer of D,L-lactic acid and 1,4-dioxane-2-one, M is Na, K or Li; and a is an integer of from 1 to 4.

YO—[C(O)—(CHX)ₐ—O-]ₘ—C(O)—R—C(O)—[—O—(CHX′)_{b}—C(O)—]ₙ—OZ [Formula 6]

In Formula 6 above, X and X′ are independently hydrogen, C₁₋₁₀ alkyl or C₆₋₂₀ aryl; Y and Z are independently Na, K or Li; m and n are independently an integer of from 0 to 95, with the proviso that 5<m+n<100; a and b are independently an integer of from 1 to 6; and R is —(CH₂)ₖ—, C₂₋₁₀ divalent alkenyl, C₆₋₂₀ divalent aryl or a combination thereof, wherein k is an integer of from 0 to 10.

The salt of polylactic acid is preferably the compound of Formula 1 or Formula 2.

In an embodiment, in order to increase the delivery efficiency of the anionic drug into cells, the composition of the present invention may further comprise a fusogenic lipid in an amount of 0.01 to 50% by weight, more specifically 0.1 to 10% by weight, based on total weight of the composition.

The fusogenic lipid, when it is mixed with the complex of the anionic drug and the cationic lipid, is combined with the complex by hydrophobic interaction to form a complex of the anionic drug, the cationic lipid and the fusogenic lipid, and the complex containing the fusogenic lipid is entrapped in the nanoparticle structure of the amphiphilic block copolymer. In an embodiment, the fusogenic lipid may be one or a combination of two or more selected from the group consisting of phospholipid, cholesterol and tocopherol.

Specifically, the phospholipid may be one or more selected from the group consisting of phosphatidylethanolamin (PE), phosphatidylcholine (PC) and phosphatidic acid. The phosphatidylethanolamin (PE), phosphatidylcholine (PC) and phosphatidic acid may be in a form combined with one or two C₁₀₋₂₄ fatty acids. The cholesterol and tocopherol include analogues, derivatives and metabolites of each of the cholesterol and tocopherol.

Specifically, the fusogenic lipid may be one or a combination of two or more selected from the group consisting of dilauroyl phosphatidylethanolamine, dimyristoyl phosphatidylethanolamine, dipalmitoyl phosphatidylethanolamine, distearoyl phosphatidylethanolamine, dioleoyl phosphatidylethanolamine, dilinoleoyl phosphatidylethanolamine, 1-palmitoyl-2-oleoyl phosphatidylethanolamine, 1,2-diphytanoyl-3-sn-phosphatidylethanolamine, dilauroyl phosphatidylcholine, dimyristoyl phosphatidylcholine, dipalmitoyl phosphatidylcholine, distearoyl phosphatidylcholine, dioleoyl phosphatidylcholine, dilinoleoyl phosphatidylcholine, 1-palmitoyl-2-oleoyl phosphatidylcholine, 1,2-diphytanoyl-3-sn-phosphatidylcholine, dilauroyl phosphatidic acid, dimyristoyl phosphatidic acid, dipalmitoyl phosphatidic acid, distearoyl phosphatidic acid, dioleoyl phosphatidic acid, dilinoleoyl phosphatidic acid, 1-palmitoyl-2-oleoyl phosphatidic acid, 1,2-diphytanoyl-3-sn-phosphatidic acid, cholesterol and tocopherol.

In a preferred embodiment, the fusogenic lipid may be one or more selected from the group consisting of dioleoyl phosphatidylethanolamine (DOPE), dipalmitooleoylphosphocholine (1,2-dipalmitoleoyl-sn-glycero-3-phosphocholine, DPPC), dioleoylphosphocholine (1,2-dioleoyl-sn-glycero-3-phosphocholine, DOPC) and dipalmitooleoylphosphoethanolamine (1,2-dipalmitoleoyl-sn-glycero-3-phosphoethanolamine, DPPE), etc.

As a concrete embodiment, the composition according to the present invention—which comprises the anionic drug-cationic compound complex entrapped in the nanoparticle structure of the amphiphilic block copolymer and the salt of polylactic acid—may be administered in the route of blood vessel, muscle, subcutaneous, oral, bone, transdermal or local tissue, and the like, and it may be formulated into various formulations for oral or parenteral administration. Examples of the formulation for oral administration may include tablet, capsule, powder, liquid, etc. and the examples of the formulation for parenteral administration may include eye drop, injection, etc. As a preferred embodiment, the composition may be a formulation for injection. For example, a freeze-dried product of the composition according to the present invention may be prepared in a form of formulation for injection by reconstituting it with distilled water for injection, 0.9% physiological saline, 5% dextrose aqueous solution, or the like.

### Effects of the Invention

According to the present invention, a composition and a method for freeze-drying of a composition for delivering anionic drug, which can allow the composition for delivering anionic drug to maintain good stability, safety and efficacy, can be provided.

### BRIEF EXPLANATION OF THE DRAWINGS

FIG. 1 is a schematic structure of the polymer nanoparticle delivery system according to an embodiment of the present invention, in which a complex of the anionic drug and the cationic compound is entrapped.

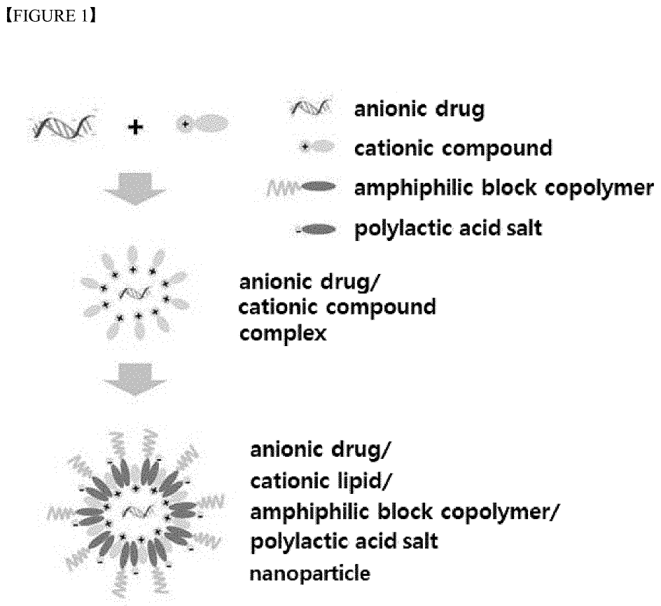

### BEST MODES FOR CARRYING OUT THE INVENTION

The present invention will be explained below in more detail with reference to the following Examples. However, the Examples are only to illustrate the invention, and the scope of the present invention is not limited thereby in any manner.

### [Preparation Example] Preparation of Polymeric Nanoparticles Containing KRAS siRNA/1,6-dioleoyl triethylenetetramide (dioTETA)/mPEG-PLA-tocopherol/PLANa/DOPE

5 μg of KRAS siRNA was dissolved in 94.52 μl of distilled water, and 94.52 μg of dioTETA was dissolved in 94.52 μl of 20 mM acetate buffer (pH 4.6), and the solutions were mixed dropwise in sonicated state. The resulting mixture was freeze-dried to a powdery state, and the powder was dissolved in with 10 μl of ethyl acetate. To this, a solution of 300 μg of PLANa dissolved in 15 μl of ethyl acetate, a solution of 104.2 μg of DOPE dissolved in 5.2 μl of ethyl acetate, and a solution of 1000 μg of mPEG-PLA-tocopherol dissolved in 20 of ethyl acetate were added in this order, and mixed. While adding the resulting mixture dropwise to 100 μl of distilled water, a complex emulsion was prepared by using a sonicator. The prepared complex emulsion was put into a 1-necked round flask and distilled under reduced pressure in a rotary evaporator for selective removal of ethyl acetate, to prepare polymeric nanoparticles containing siRNA/1,6-dioleoyl triethylenetetramide (dioTETA)/mPEG-PLA-tocopherol/PLANa/DOPE.

TABLE 1

| | | | | Cationic | Polymer | Polymer | Helper |
|---|---|---|---|---|---|---|---|
| | Composition | Ratio | siRNA | lipid | 1 | 2 | lipid |
| Preparation | siRNA/dioTETA/mPEG-PLA- | 5-18-1- | 5 μg | 94.5 μg | 1000 μg | 300 μg | 104.2 μg |
| Example 1 | tocopherol/PLANa/DOPE | 0.3-1 | | | | | |

### [Examples 1 to 4] Preparation of Freeze-Dried Formulation of Polymeric Nanoparticles Containing KRAS siRNA/1,6-Dioleoyl Triethylenetetramide (dioTETA)/mPEG-PLA-Tocopherol/PLANa/DOPE/Sorbitol

The concentration of the polymeric nanoparticle prepared in Preparation Example was fixed to 100 ng/μl of siRNA and sorbitol was added thereto, and the mixture was frozen in an ultradeep freezer, and then the freeze-drying was conducted. The freeze-drying conditions were the same as provided in the following Table 2.

TABLE 2

| | Temperature condition | | Vacuum | Process |
|---|---|---|---|---|
| | Set temper- | Mode for | degree | time |
| Step | ature (° C.) | change | (mTorr) | (min.) |
| Initial freezing | −40 | Lowering | Atmospheric | 90 |
| Freezing | −40 | Maintaining | Atmospheric | 240 |
| Cold Trap | −50 | Lowering | Atmospheric | 20 |
| 1 Step | −40 | Maintaining | 200 | 60 |
| 2 Step | −10 | Elevating | 200 | 240 |
| 3 Step | −10 | Maintaining | 200 | 360 |
| 4 Step | +5 | Elevating | 200 | 120 |
| 5 Step | +5 | Maintaining | 150 | 1,200 |
| 6 Step | +25 | Elevating | 150 | 120 |
| 7 Step | +25 | Maintaining | 150 | 240 |

| | |
|---|---|
| Total | 2,690 |

To the freeze-dried powder, sterilized distilled water at room temperature was added and the mixture was shaken to dissolve the powder, to prepare a composition containing siRNA/dio-TETA/mPEG-PLA-tocopherol/PLANa/DOPE/sorbitol.

TABLE 3

| | | | | Cationic | Polymer | Polymer | Helper | |
|---|---|---|---|---|---|---|---|---|
| | Composition | Ratio | siRNA | lipid | 1 | 2 | lipid | Sorbitol |
| Example | siRNA/dioTETA/mPEG-PLA- | 5-18-1- | 5 μg | 94.5 μg | 1000 μg | 300 μg | 104.2 μg | 0.25 mg |
| 1 | tocopherol/PLANa/DOPE/sorbitol | 0.3-1 | | | | | | |
| Example | siRNA/dioTETA/mPEG-PLA- | 5-18-1- | 5 μg | 94.5 μg | 1000 μg | 300 μg | 104.2 μg | 2.5 mg |
| 2 | tocopherol/PLANa/DOPE/sorbitol | 0.3-1 | | | | | | |
| Example | siRNA/dioTETA/mPEG-PLA- | 5-18-1- | 5 μg | 94.5 μg | 1000 μg | 300 μg | 104.2 μg | 5 mg |
| 3 | tocopherol/PLANa/DOPE/sorbitol | 0.3-1 | | | | | | |
| Example | siRNA/dioTETA/mPEG-PLA- | 5-18-1- | 5 μg | 94.5 μg | 1000 μg | 300 μg | 104.2 μg | 10 mg |
| 4 | tocopherol/PLANa/DOPE/sorbitol | 0.3-1 | | | | | | |

### [Comparative Examples 1 to 3] Preparation of Freeze-Dried Formulation of Polymeric Nanoparticles Containing KRAS siRNA/1,6-Dioleoyl Triethylenetetramide (dioTETA)/mPEG-PLA-Tocopherol/PLANa/DOPE/Trehalose

By using trehalose as a cryoprotectant, polymeric nanoparticles containing siRNA/dio-TETA/mPEG-PLA-tocopherol/PLANa/DOPE/trehalose were prepared in the same manner as described in Example 1.

TABLE 4

| | | | | Cationic | Polymer | Polymer | Helper | |
|---|---|---|---|---|---|---|---|---|
| | Composition | Ratio | siRNA | lipid | 1 | 2 | lipid | Trehalose |
| Comp. | siRNA/dioTETA/mPEG-PLA- | 5-18-1- | 5 μg | 94.5 μg | 1000 μg | 300 μg | 104.2 μg | 2.5 mg |
| Example | tocopherol/PLANa/DOPE/trehalose | 0.3-1 | | | | | | |
| 1 | | | | | | | | |
| Comp. | siRNA/dioTETA/mPEG-PLA- | 5-18-1- | 5 μg | 94.5 μg | 1000 μg | 300 μg | 104.2 μg | 5 mg |
| Example | tocopherol/PLANa/DOPE/trehalose | 0.3-1 | | | | | | |
| 2 | | | | | | | | |
| Comp. | siRNA/dioTETA/mPEG-PLA- | 5-18-1- | 5 μg | 94.5 μg | 1000 μg | 300 μg | 104.2 μg | 10 mg |
| Example | tocopherol/PLANa/DOPE/trehalose | 0.3-1 | | | | | | |
| 3 | | | | | | | | |

### [Comparative Example 4] Preparation of Freeze-Dried Formulation of Polymeric Nanoparticles Containing KRAS siRNA/1,6-Dioleoyl Triethylenetetramide (dioTETA)/mPEG-PLA-Tocopherol/PLANa/DOPE/Mannitol

By using mannitol as a cryoprotectant, polymeric nanoparticles containing siRNA/dio-TETA/mPEG-PLA-tocopherol/PLANa/DOPE/mannitol were prepared in the same manner as described in Example 1.

TABLE 5

| | | | | Cationic | Polymer | Polymer | Helper | |
|---|---|---|---|---|---|---|---|---|
| | Composition | Ratio | siRNA | lipid | 1 | 2 | lipid | Mannitol |
| Comp. | siRNA/dioTETA/mPEG-PLA- | 5-18-1- | 5 μg | 94.5 μg | 1000 μg | 300 μg | 104.2 μg | 2.5 mg |
| Example | tocopherol/PLANa/DOPE/mannitol | 0.3-1 | | | | | | |
| 4 | | | | | | | | |

### [Comparative Example 5] Preparation of Freeze-Dried Formulation of Polymeric Nanoparticles Containing KRAS siRNA/1,6-Dioleoyl Triethylenetetramide (dioTETA)/mPEG-PLA-Tocopherol/PLANa/DOPE/Sucrose

By using sucrose as a cryoprotectant, polymeric nanoparticles containing siRNA/dio-TETA/mPEG-PLA-tocopherol/PLANa/DOPE/sucrose were prepared in the same manner as described in Example 1.

TABLE 6

| | | | | Cationic | Polymer | Polymer | Helper | |
|---|---|---|---|---|---|---|---|---|
| | Composition | Ratio | siRNA | lipid | 1 | 2 | lipid | Sucrose |
| Comp. | siRNA/dioTETA/mPEG-PLA- | 5-18-1- | 5 μg | 94.5 μg | 1000 μg | 300 μg | 104.2 μg | 2.5 mg |
| Example | tocopherol/PLANa/DOPE/sucrose | 0.3-1 | | | | | | |
| 5 | | | | | | | | |

### [Comparative Example 6] Preparation of Freeze-Dried Formulation of Polymeric Nanoparticles Containing KRAS siRNA/1,6-Dioleoyl Triethylenetetramide (dioTETA)/mPEG-PLA-Tocopherol/PLANa/DOPE/Glucose

By using glucose as a cryoprotectant, polymeric nanoparticles containing siRNA/dio-TETA/mPEG-PLA-tocopherol/PLANa/DOPE/glucose were prepared in the same manner as described in Example 1.

TABLE 7

| | | | | Cationic | Polymer | Polymer | Helper | |
|---|---|---|---|---|---|---|---|---|
| | Composition | Ratio | siRNA | lipid | 1 | 2 | lipid | Glucose |
| Comp. | siRNA/dioTETA/mPEG-PLA- | 5-18-1- | 5 μg | 94.5 μg | 1000 μg | 300 μg | 104.2 μg | 2.5 mg |
| Example | tocopherol/PLANa/DOPE/glucose | 0.3-1 | | | | | | |
| 6 | | | | | | | | |

### [Experimental Example 1] Size Measurement and Stability Comparison (Heparin Competition Analysis) for Polymeric Nanoparticles Containing siRNA/dioTETA/mPEG-PLA-Tocopherol/PLANa/DOPE/Cryoprotectant

Size measurement was conducted for the polymeric nanoparticles prepared with different cryoprotectants. The sizes of the particles were measured by using Dynamic Light Scattering (DLS) method. Specifically, a He—Ne laser was used as a light source, and Zetasizer Nano ZS90 (MALVERN) device was operated according to the manual.

In addition, heparin competition analysis was conducted to evaluate in vivo stability of the polymeric nanoparticles according to the kind of cryoprotectant. 10 μl of each formulation (siRNA 300 ng) was treated with 40 μg of heparin and reacted for 10 minutes at room temperature, and then the amount of disintegrated siRNA was measured through electrophoresis. The lower disintegration degree of siRNA indicates the better stability of the formulation. In addition, the formulation itself alone was subjected to electrophoresis to measure the amount of unentrapped siRNA in the formulation.

The measured results of the unentrapped siRNA, particle size, and disintegration degree of siRNA for the formulations of Example 2 and Comparative Examples 1 and 4 to 6 prepared with different cryoprotectants are shown in the following Table 8.

TABLE 8

| | | Unentrapped | Particle | Disintegration |
|---|---|---|---|---|
| | Composition | siRNA | size | degree of siRNA (%) |
| Preparation | Aqueous solution formulation of | 0% | 38 nm | 5% |
| Example | siRNA/dioTETA/mPEG-PLA- | | | |
| | tocopherol/PLANa/DOPE | | | |
| Example | Freeze-dried formulation of | 0% | 46 nm | 13% |
| 2 | siRNA/dioTETA/mPEG-PLA- | | | |
| | tocopherol/PLANa/DOPE/ | | | |
| | sorbitol 2.5 mg | | | |
| Comp. | Freeze-dried formulation of | 5% | 23 nm | 8% |
| Example | siRNA/dioTETA/mPEG-PLA- | | | |
| 1 | tocopherol/PLANa/DOPE/ | | | |
| | trehalose 2.5 mg | | | |
| Comp. | Freeze-dried formulation of | 18% | 25 nm | 14% |
| Example | siRNA/dioTETA/mPEG-PLA- | | | |
| 4 | tocopherol/PLANa/DOPE/ | | | |
| | mannitol 2.5 mg | | | |
| Comp. | Freeze-dried formulation of | 0% | 154 nm | 37% |
| Example | siRNA/dioTETA/mPEG-PLA- | | | |
| 5 | tocopherol/PLANa/DOPE/ | | | |
| | sucrose 2.5 mg | | | |
| Comp. | Freeze-dried formulation of | 0% | 34 nm | 15% |
| Example | siRNA/dioTETA/mPEG-PLA- | | | |
| 6 | tocopherol/PLANa/DOPE/ | | | |
| | glucose 2.5 mg | | | |

As can be know from Table 8, in case of using trehalose or mannitol, the amount of unentrapped siRNA increased, and in case of using sucrose, the disintegration degree of siRNA was high. Thus, it could be confirmed that trehalose, mannitol and sucrose are not suitable cryoprotectants.

The analysis results for the formulations of Examples 1 to 4 and Comparative Examples 1 to 3 prepared with different amounts of sorbitol or trehalose among the cryoprotectants are shown in the following Table 9.

TABLE 9

| | | Unentrapped | Particle | Disintegration |
|---|---|---|---|---|
| | Composition | siRNA | size | degree of siRNA (%) |
| Preparation | Aqueous solution formulation of | 0% | 38 nm | 5% |
| Example | siRNA/dioTETA/mPEG-PLA- | | | |
| | tocopherol/PLANa/DOPE | | | |
| Example | Freeze-dried formulation of | 0% | 42 nm | 16% |
| 1 | siRNA/dioTETA/mPEG-PLA- | | | |
| | tocopherol/PLANa/DOPE/ | | | |
| | sorbitol 0.25 mg | | | |
| Example | Freeze-dried formulation of | 0% | 46 nm | 13% |
| 2 | siRNA/dioTETA/mPEG-PLA- | | | |
| | tocopherol/PLANa/DOPE/ | | | |
| | sorbitol 2.5 mg | | | |
| Example | Freeze-dried formulation of | 0% | 48 nm | 8% |
| 3 | siRNA/dioTETA/mPEG-PLA- | | | |
| | tocopherol/PLANa/DOPE/ | | | |
| | sorbitol 5 mg | | | |
| Example | Freeze-dried formulation of | 0% | 34 nm | 13% |
| 4 | siRNA/dioTETA/mPEG-PLA- | | | |
| | tocopherol/PLANa/DOPE/ | | | |
| | sorbitol 10 mg | | | |
| Comp. | Freeze-dried formulation of | 5% | 23 nm | 8% |
| Example | siRNA/dioTETA/mPEG-PLA- | | | |
| 1 | tocopherol/PLANa/DOPE/ | | | |
| | trehalose 2.5 mg | | | |
| Comp. | Freeze-dried formulation of | 25% | 43 nm | 28% |
| Example | siRNA/dioTETA/mPEG-PLA- | | | |
| 2 | tocopherol/PLANa/DOPE/ | | | |
| | trehalose 5 mg | | | |
| Comp. | Freeze-dried formulation of | 14% | 52 nm | 26% |
| Example | siRNA/dioTETA/mPEG-PLA- | | | |
| 3 | tocopherol/PLANa/DOPE/ | | | |
| | trehalose 10 mg | | | |

As can be known from Table 9, in case of using trehalose, the amount of unentrapped siRNA increased, and the disintegration degree of siRNA became higher as the amount of trehalose increased. Thus, it could be confirmed that trehalose is not a suitable cryoprotectant.

### [Experimental Example 2] Cell Efficacy and Toxicity Comparison of Polymeric Nanoparticles Containing siRNA/dioTETA/mPEG-PLA-Tocopherol/PLANa/DOPE/Cryoprotectant

For the polymeric nanoparticles containing siRNA/dioTETA/mPEG-PLA-tocopherol/PLANa/DOPE/cryoprotectant prepared in Preparation Example, Examples 2 to 4 and Comparative Examples 1 and 6, the efficacy of delivering siRNA to A549 lung cancer cell line was evaluated in mRNA level. The cells were seeded to a 96-well cell culture plate at 5000 cells/well concentration. After 24 hours, it was confirmed that about 50 to 60% of cells in each well were grown uniformly. Then, the medium in the well was removed, and 90 μl of fresh medium containing serum at 10% of final volume was added. To the cell culture medium, each of the compositions of Preparation Example, Examples 2 to 4 and Comparative Examples 1 and 6 was added so that siRNA might be contained at 400 nM, 200 nM, 100 nM, 50 nM, 5 nM, 0.5 nM, 0.05 nM concentration. The cells were cultured in an incubator at 37° C. with 5% CO₂ for 48 hours and the medium was removed, and then 100 μl of cell lysis mixture was added and reacted at 50° C. for 18 hours. Thereafter, in order to evaluate mRNA expression, Branched DNA assay (bDNA, Quantigene 2.0 Assay kit, Panomics, QS0009) was used. According to the protocol, 2.0 substrate was added and reacted at room temperature for 5 minutes, and then microplate fluorescence reader (Bio-Tek, Synergy HT) was used to measure the fluorescence expression amount. Furthermore, in order to analyze intracellular toxicity, Cell Titer-Glo luminescent cell viability assay (Promega, G7571) was used. According to the protocol, to the cell plate at room temperature, the sample for analysis (100 μl) and a cell titer assay reagent (100 μl) were added and reacted for 30 minutes, and then microplate fluorescence reader (Bio-Tek, Synergy HT) was used to measure the value. The results of evaluating efficacy and toxicity for the cell line are shown in the following Table 10.

| | Composition | LC₅₀ | IC₅₀ |
|---|---|---|---|
| Preparation | Aqueous solution formulation of | 138 | 32.3 |
| Example | siRNA/dioTETA/mPEG-PLA- | | |
| | tocopherol/PLANa/DOPE | | |
| Example | Freeze-dried formulation of | 262 | 22.1 |
| 2 | siRNA/dioTETA/mPEG-PLA- | | |
| | tocopherol/PLANa/DOPE/ | | |
| | sorbitol 2.5 mg | | |
| Example | Freeze-dried formulation of | 290.7 | 11.2 |
| 3 | siRNA/dioTETA/mPEG-PLA- | | |
| | tocopherol/PLANa/DOPE/ | | |
| | sorbitol 5 mg | | |
| Example | Freeze-dried formulation of | 238.8 | 9.0 |
| 4 | siRNA/dioTETA/mPEG-PLA- | | |
| | tocopherol/PLANa/DOPE/ | | |
| | sorbitol 10 mg | | |
| Comp. | Freeze-dried formulation of | 293.6 | 4.6 |
| Example | siRNA/dioTETA/mPEG-PLA- | | |
| 1 | tocopherol/PLANa/DOPE/ | | |
| | trehalose 2.5 mg | | |
| Comp. | Freeze-dried formulation of | 179.9 | 6.3 |
| Example | siRNA/dioTETA/mPEG-PLA- | | |
| 2 | tocopherol/PLANa/DOPE/ | | |
| | glucose 2.5 mg | | |

As clearly described in Table 10, the results showed the tendency that the freeze-drying reduced the toxicity and improved the efficacy since after the freeze-drying, LC₅₀ was increased and IC₅₀ was decreased, as compared with those before the freeze-drying. As a result of calculating LC₅₀/IC₅₀, the samples showed the efficacy in the order of sorbitol 2.5 mg>sorbitol 5 mg>sorbitol 10 mg>glucose 2.5 mg>trehalose 2.5 mg.

1. A composition for freeze-drying of a composition for delivering anionic drug, which comprises:
a composition for delivering anionic drug comprising an anionic drug as an active ingredient, a cationic compound, an amphiphilic block copolymer and one or more salts of polylactic acid selected from the group consisting of the compounds of the following Formulas 1 to 6, wherein the anionic drug forms a complex with the cationic compound by electrostatic interaction, the complex is entrapped in a nanoparticle structure formed by the amphiphilic block copolymer and the salt of polylactic acid; and
sorbitol as a cryoprotectant:
RO—CHZ-[A]ₙ-[B]ₘ—COOM [Formula 1]
wherein A is —COO—CHZ—; B is —COO—CHY—, —COO—CH₂CH₂CH₂CH₂CH₂— or —COO—CH₂CH₂OCH₂; R is a hydrogen atom, or acetyl, benzoyl, decanoyl, palmitoyl, methyl or ethyl; each of Z and Y is a hydrogen atom, or methyl or phenyl; M is Na, K or Li; n is an integer of from 1 to 30; and m is an integer of from 0 to 20;
RO—CHZ—[COO—CHX]ₚ—[COO—CHY′]_{g}—COO—CHZ—COOM [Formula 2]
wherein X is methyl; Y′ is a hydrogen atom or phenyl; p is an integer of from 0 to 25, q is an integer of from 0 to 25, with the proviso that p+q is an integer of from 5 to 25; R is a hydrogen atom, or acetyl, benzoyl, decanoyl, palmitoyl, methyl or ethyl; M is Na, K or Li; and Z is a hydrogen atom, methyl or phenyl;
RO-PAD-COO—W-M′ [Formula 3]
wherein W-M′ is 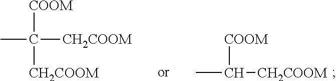
PAD is selected from the group consisting of D,L-polylactic acid, D-polylactic acid, polymandelic acid, copolymer of D,L-lactic acid and glycolic acid, copolymer of D,L-lactic acid and mandelic acid, copolymer of D,L-lactic acid and caprolactone, and copolymer of D,L-lactic acid and 1,4-dioxane-2-one; R is a hydrogen atom, or acetyl, benzoyl, decanoyl, palmitoyl, methyl or ethyl; and M is independently Na, K or Li;
S—O-PAD-COO-Q [Formula 4]
wherein S is 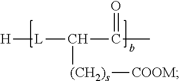
L is —NR₁— or —O—, wherein R₁ is a hydrogen atom or C₁₋₁₀ alkyl; Q is CH₃, CH₂CH₃, CH₂CH₂CH₃, CH₂CH₂CH₂CH₃, or CH₂C₆H₅; a is an integer of from 0 to 4; b is an integer of from 1 to 10; M is Na, K or Li; and PAD is one or more selected from the group consisting of D,L-polylactic acid, D-polylactic acid, polymandelic acid, copolymer of D,L-lactic acid and glycolic acid, copolymer of D,L-lactic acid and mandelic acid, copolymer of D,L-lactic acid and caprolactone, and copolymer of D,L-lactic acid and 1,4-dioxane-2-one; 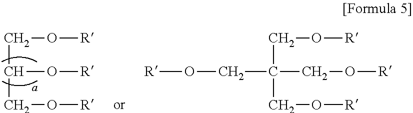
wherein R′ is -PAD-O—C(O)—CH₂CH₂—C(O)—OM, wherein PAD is selected from the group consisting of D,L-polylactic acid, D-polylactic acid, polymandelic acid, copolymer of D,L-lactic acid and glycolic acid, copolymer of D,L-lactic acid and mandelic acid, copolymer of D,L-lactic acid and caprolactone, and copolymer of D,L-lactic acid and 1,4-dioxane-2-one, M is Na, K or Li; and a is an integer of from 1 to 4;
YO—[C(O)—(CHX)ₐ—O-]ₘ—C(O)—R—C(O)—[—O—(CHX′)_{b}—C(O)—]ₙ—OZ [Formula 6]
wherein X and X′ are independently hydrogen, C₁-10 alkyl or C₆₋₂₀ aryl; Y and Z are independently Na, K or Li; m and n are independently an integer of from 0 to 95, with the proviso that 5<m+n<100; a and b are independently an integer of from 1 to 6; and R is —(CH₂)ₖ—, C₂₋₁₀ divalent alkenyl, C₆₋₂₀ divalent aryl or a combination thereof, wherein k is an integer of from 0 to 10.

2. The composition for freeze-drying of a composition for delivering anionic drug of claim 1, which comprises the sorbitol in an amount of 1 to 5,000 parts by weight, based on 1 part by weight of the anionic drug.

3. The composition for freeze-drying of a composition for delivering anionic drug of claim 1, wherein the anionic drug is nucleic acid.

4. The composition for freeze-drying of a composition for delivering anionic drug of claim 1, wherein the cationic compound is one or more selected from the group consisting of cationic lipids and cationic polymers.

5. The composition for freeze-drying of a composition for delivering anionic drug of claim 4, wherein the cationic lipid is one or more selected from the group consisting of N,N-dioleyl-N,N-dimethylammoniumchloride (DODAC), N,N-distearyl-N,N-dimethylammoniumbromide (DDAB), N-(1-(2,3-dioleoyloxy)propyl-N,N,N-trimethylammoniumchloride (DOTAP), N,N-dimethyl-(2,3-dioleoyloxy)propylamine (DODMA), 1,2-diacyl-3-trimethylammonium-propane (TAP), 1,2-diacyl-3-dimethylammonium-propane (DAP), 3β-[N—(N′,N′,N′-trimethylaminoethane)carbamoyl]cholesterol (TC-cholesterol), 3β[N—(N′,N′-dimethylaminoethane)carbamoyl]cholesterol (DC-cholesterol), 3β-[N—(N′-monomethylaminoethane)carbamoyl]cholesterol (MC-cholesterol), 3β-[N-(aminoethane)carbamoyl]cholesterol (AC-cholesterol), cholesteryloxypropane-1-amine (COPA), N—(N′-aminoethane)carbamoylpropanoic tocopherol (AC-tocopherol) and N—(N′-methylaminoethane)carbamoylpropanoic tocopherol (MC-tocopherol).

6. The composition for freeze-drying of a composition for delivering anionic drug of claim 4, wherein the cationic lipid is a cationic lipid represented by the following Formula 7: 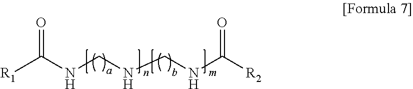
wherein each of n and m is 0 to 12 with the proviso that 2≤n+m≤12, each of a and b is 1 to 6, and each of R₁ and R₂ is independently selected from the group consisting of saturated and unsaturated C₁₁₋₂₅ hydrocarbons.

7. The composition for freeze-drying of a composition for delivering anionic drug of claim 6, wherein each of R₁ and R₂ is independently selected from the group consisting of lauryl, myristyl, palmityl, stearyl, arachidyl, behenyl, lignoceryl, cerotyl, myristoleyl, palmitoleyl, sapienyl, oleyl, linoleyl, arachidonyl, eicosapentaenyl, erucyl, docosahexaenyl and cerotyl.

8. The composition for freeze-drying of a composition for delivering anionic drug of claim 1, wherein the amphiphilic block copolymer is an A-B type di-block copolymer comprising a hydrophilic A block and a hydrophobic B block, wherein the hydrophilic A block is one or more selected from the group consisting of polyalkyleneglycol, polyvinyl alcohol, polyvinyl pyrrolidone, polyacrylamide and derivatives thereof, and the hydrophobic B block is one or more selected from the group consisting of polyester, polyanhydride, polyamino acid, polyorthoester and polyphosphazine.

9. The composition for freeze-drying of a composition for delivering anionic drug of claim 8, wherein a hydroxyl group at the end of the hydrophobic B block is modified by one or more selected from the group consisting of cholesterol, tocopherol and C₁₀₋₂₄ fatty acid.

10. The composition for freeze-drying of a composition for delivering anionic drug of claim 1, further comprising a fusogenic lipid.

11. The composition for freeze-drying of a composition for delivering anionic drug of claim 10, wherein the fusogenic lipid is one or more selected from the group consisting of dilauroyl phosphatidylethanolamine, dimyristoyl phosphatidylethanolamine, dipalmitoyl phosphatidylethanolamine, distearoyl phosphatidylethanolamine, dioleoyl phosphatidylethanolamine, dilinoleoyl phosphatidylethanolamine, 1-palmitoyl-2-oleoyl phosphatidylethanolamine, 1,2-diphytanoyl-3-sn-phosphatidylethanolamine, dilauroyl phosphatidylcholine, dimyristoyl phosphatidylcholine, dipalmitoyl phosphatidylcholine, distearoyl phosphatidylcholine, dioleoyl phosphatidylcholine, dilinoleoyl phosphatidylcholine, 1-palmitoyl-2-oleoyl phosphatidylcholine, 1,2-diphytanoyl-3-sn-phosphatidylcholine, dilauroyl phosphatidic acid, dimyristoyl phosphatidic acid, dipalmitoyl phosphatidic acid, distearoyl phosphatidic acid, dioleoyl phosphatidic acid, dilinoleoyl phosphatidic acid, 1-palmitoyl-2-oleoyl phosphatidic acid, 1,2-diphytanoyl-3-sn-phosphatidic acid, cholesterol and tocopherol.

12. A method for freeze-drying of a composition for delivering anionic drug, comprising conducting the freeze-drying by using a composition according to claim 1.

13. A freeze-dried product of a composition for delivering anionic drug freeze-dried by the method according to claim 12.
